(19) United States
(12) Patent Application Publication
(10) Pub. No.: US 2026/0105050 A1
(43) Pub. Date: Apr. 16, 2026

(54) DYNAMIC MIXED ROUTING IN LLMS FOR MEDICAL DECISION MAKING

(71) Applicant: NEC Laboratories America, Inc., Princeton, NJ (US)

(72) Inventors: Yanchi Liu, Princeton, NJ (US); Wenchao Yu, Plainsboro, NJ (US); Wei Cheng, Princeton Junction, NJ (US); Haifeng Chen, West Windsor, NJ (US); Xujiang Zhao, Hillsborough, NJ (US); Zhengzhang Chen, Princeton Junction, NJ (US); Xinyuan Wang, Tempe, AZ (US)

(21) Appl. No.: 19/353,843

(22) Filed: Oct. 9, 2025

Related U.S. Application Data
(60) Provisional application No. 63/706,273, filed on Oct. 11, 2024.

(51) Int. Cl. G06F 16/2453; G16H 10/60; G16H 20/00; G16H 40/20

(52) U.S. Cl. CPC G06F 16/2453; G16H 10/60; G16H 20/00; G16H 40/20

(57) ABSTRACT

Methods and systems include embedding an input query, including contextual information. Performance and cost of executing the input query are predicted on each of a set of language models. The prediction is performed using a multi-armed bandit approach with each of the language models being represented by a respective arm. The input query is executed on a selected model that has a best balance of performance and cost.

### RELATED APPLICATION INFORMATION

This application claims priority to U.S. Patent Application No. 63/706,273, filed on Oct. 11, 2024, incorporated herein by reference in its entirety.

### BACKGROUND

### Technical Field

The present invention relates to large language models (LLMs) and, more particularly, to dynamic mixed routing in LLMs.

### Description of the Related Art

As LLMs increase in model size, this has generally led to improvements in quality. LLMs have achieved superior performance not only in natural language processing tasks but also in other fields. Currently, LLMs are being used as universal models for multiple tasks. Multi-task capability has become a key metric for evaluating an LLM. However, there are many LLMs available, each with its own strengths and weaknesses.

Fine-tuning or merely running inference on an LLM is very costly due to their large size. Selecting between suitable pretrained LLMs for specific queries is needed to provide the best results at a low cost.

### SUMMARY

A method includes embedding an input query, including contextual information. Performance and cost of executing the input query are predicted on each of a set of language models. The prediction is performed using a multi-armed bandit approach with each of the language models being represented by a respective arm. The input query is executed on a selected model that has a best balance of performance and cost.

A system includes a hardware processor and a memory that stores a computer program. When executed by the hardware processor, causes the hardware processor to embed an input query, including contextual information, to predict performance and cost of executing the input query on each of a plurality of language models, the prediction being performed using a multi-armed bandit approach with each of the language models being represented by a respective arm, and to execute the input query on a selected model of the plurality of models that has a best balance of performance and cost.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of dynamic query routing, in accordance with an embodiment of the present invention;

FIG. 2 is a block/flow diagram of a method for training and using a dynamic query routing selector, in accordance with an embodiment of the present invention;

FIG. 3 is a block diagram of a healthcare facility that uses dynamic query routing, in accordance with an embodiment of the present invention;

FIG. 4 is a block diagram of a computing device that trains and uses a query routing selector, in accordance with an embodiment of the present invention;

FIG. 5 is a diagram of an exemplary neural network architecture that can be used to implement part of a model selector, in accordance with an embodiment of the present invention; and

FIG. 6 is a diagram of an exemplary neural network architecture that can be used to implement part of a model selector, in accordance with an embodiment of the present invention.

### DETAILED DESCRIPTION OF PREFERRED EMBODIMENTS

Selecting between available large language models (LLMs) can be a challenging balance between their power and their cost. For example, some powerful LLMs may deliver superior performance, but may incur high economic and computational costs and may further suffer from high latency. On the other hand, selecting a relatively small LLM may reduce cost and increase the speed of execution, but the outputs of the LLM may be less reliable.

To that end, query-level routing may be used that efficiently selects the most suitable LLM for each query based on a prediction of performance, cost, and latency. A contextual multi-armed bandit (MAB) approach is used to embed queries and LLM-specific statements and to predict performance and response length. A meta-decision-making process chooses the best LLM for each query. This avoids the need to test multiple models during inference and provides rapid adaptation to available LLMs. Domain-specific tags may be used to enhance the embeddings, improving the accuracy of the routing model. The prediction is enhanced by an understanding of the context of each query.

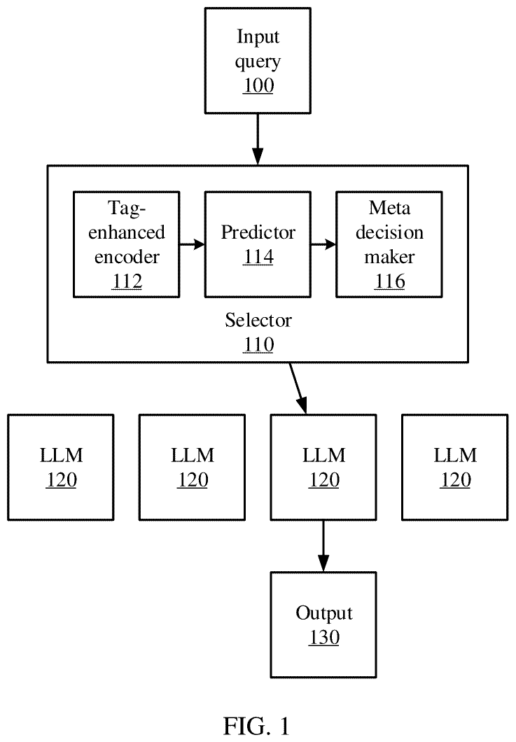

Referring now to FIG. 1, an LLM selection system is shown. A selector 110 receives an input query 100. The selector 110 considers multiple different LLMs 120, each having different properties. For example, each of the LLMs 120 may have a different number of parameters and may be pretrained for particular domains or particular functions. Based on a balance of execution cost, latency, and expected output quality, the selector 110 picks one of the LLMs 120 and sends the input query 100 to it. The selected LLM 120 then processes the input query 100 to generate an output 130, which may be used for some downstream task. During operation, LLMs 120 may be added or removed from the pool. This does not necessitate a complete re-training.

The selector 110 performs query-level routing. Although the problem is fundamentally at the set level, the present embodiments approach it at the query level to ensure that each query is answered by a suitable LLM. Specifically, the selector 110 considers input query 100 and a set of candidate LLMs 120. The selector 110 selects one of the candidate LLMs 120 as the most suitable for the given query. The selector 110 performs this selection using a contextual MAB approach. The contextual information includes an embedding of the input query 100 and the candidate LLMs 120.

First, a tag-enhanced encoder 112 generates the query embedding, incorporating relevant contextual information. Next, predictor 114 predicts the performance and cost for executing the input query 100 by each candidate LLM 120. Finally, a meta decision maker 116 selects the most suitable LLM 120 based on the predicted values, considering the waiting time constraint and uncertainty.

The meta-decision maker 116 determines a score for the LLMs based on performance and cost predicted, considering latency and uncertainty, as:

$${s}_{n , l} = {s}_{n , l}^{trade} + α \cdot {s}_{n , l}^{unc} - β \cdot {s}_{l}^{pen}$$

where

$${s}_{n , l}^{trade}$$

is a trade-off of the predicted quality and cost for an LLM l and an n^{th} query,

$${s}_{n , l}^{unc}$$

accounts for potential prediction uncertainty,

$${s}_{l}^{p e n}$$

discourages selecting candidates with a long waiting time, and α and β control relative importance of the different terms.

To effectively route each query to the most suitable LLM, embeddings for both the query and the candidate LLMs are generated by the tag-enhanced encoder 112. For a given query qₙ, the embedding vector is obtained as:

$${e}_{n}^{q} = Encoder ( {q}_{n} )$$

Here, the encoder 112 may be implemented as a BERT (Bidirectional Encoder Representations from Transformers)-like model designed for sentence-level encoding. The model of the encoder 112 may be retrained using a specialized loss function. Tags are generated for each query, for example using the InsTag model. These tags are fine-grained, and so are cluster to represent M different domains. A composite loss function is used to maximize intra-domain similarity and minimize inter-domain similarity, defined as:

$$L = {L}_{variation} + {L}_{separation}$$

The intra-domain similarity loss Lᵥₐᵣᵢₐₜᵢₒₙ encourages the embeddings within the same domain to be close to their corresponding prototype, defined as:

$${L}_{variation} = - \frac{1}{N} {\sum }_{i = 1}^{N} \frac{exp ( {e}_{i} \cdot {p}_{{y}_{i}} )}{{Σ}_{j = 1}^{M} exp ( {e}_{i} \cdot {p}_{j} )}$$

where p_{y}_{ᵢ} is a prototype (center) of the domain which an i^{th} query belongs to, pⱼ is the prototype of the j^{th} domain, and N is the total number of queries. The tags of queries are clustered into M different domains which may, for example, represent different subject matter such as healthcare, computer technology, mathematics, etc.

The inter-domain separation loss Lₛₑₚₐᵣₐₜᵢₒₙ encourages the prototypes of different domains to be as distinct as possible:

$${L}_{separation} = \frac{1}{M} {\sum }_{J = 1}^{M} {\sum }_{k \neq j} exp ( {p}_{j} \cdot {p}_{k} )$$

In addition to the query embedding, each of the L candidate LLMs is associated with a textual statement that describes its characteristics and features:

$$States = \{ S t a t {e}_{L L {M}_{1}} , S t a t {e}_{L L {M}_{2}} , … , {State}_{{LLM}_{L}} \}$$

These statements are also embedded through the 112. For each input query 100, these embeddings are LLM-specific, meaning one query is associated with L embedding vectors, each corresponding to a different LLM 120. The final embedding for the query-LLM pair is obtained by concatenating the query embedding and the LLM state embedding:

$${e}_{n}^{l} = \frac{C o n c a t ( {e}_{n}^{q} , {e}^{l} )}{\sqrt{2}}$$

where division by \sqrt{2} serves as normalization.

To select the most suitable LLM 120 for each input query 100, the predictor 114 predicts both the performance and the cost associated with each candidate LLM 120, also referred to herein as each arm of the MAB. For performance prediction, a set of predictive functions is defined to estimate the performance of different candidate LLMs:

$${F}^{P} = \{ {f}_{1}^{P} , {f}_{2}^{P} , … , {f}_{L}^{P} \}$$

where each

$${f}_{l}^{P}$$

is an arm-specific function parameterized by

$${θ}_{l}^{P}$$

that predicts the performance score for a given embedding:

$${\hat{p}}_{n}^{l} = {f}_{l}^{P} ( {e}_{n}^{l} , {θ}_{l}^{P} )$$

These performance predictors can be implemented using various methods. The most intuitive approach is to use a regressor. However, training-free models can also be employed. For example, the performance of larger LLMs can be estimated using scaling laws derived from the performance of smaller LLMs.

Similarly, the cost associated with each LLM 120 is predicted. The cost may include two components: the input prompt cost and the output response cost. Generally, the unit price for the input prompt is lower than that for the output response.

The total cost is determined based on these two components. The input prompt length is straightforward to obtain using a token counter, as shown in the equation below. This step is not predictive, as the query length is known before calling any LLM:

$$l e n g t {h}_{n}^{l , prompt} = TokenCount ( {q}_{n} )$$

For the response part, the exact token length cannot be known before querying the LLMs, and so predictive models determine this length. A set of response length predictors, like the performance predictors, is represented as:

$${F}^{R L} = \{ {f}_{1}^{R L} , {f}_{2}^{R L} , … , {f}_{L}^{R L} \}$$

where each

$${f}_{l}^{R L}$$

is a predictive function specific to the l^{w} 120, parameterized by

$${θ}_{l}^{R L} .$$

The predicted response length for each arm is then given by:

$$l {h}_{n}^{l , response} = {f}_{l}^{R L} ( {e}_{n}^{l} , {θ}_{l}^{R L} )$$

The predictive functions may be implemented with any appropriate predictive methods.

Finally, the overall cost for each LLM 120 is computed as the sum of two components:

$${\hat{c}}_{n}^{l} = {length}_{n}^{l , response} \cdot {Price}_{l}^{prompt} + l {h}_{n}^{l , response} \cdot {Price}_{l}^{response}$$

Using these predictions and the overall cost, along with uncertainty and time penalty factors, meta decision maker 116 determines the most suitable LLM 120 for the input query 100. A score for each LLM l on the query n is determined as:

$${Score}_{n}^{l} = {r}_{n}^{l} + α \cdot {Uncertainty}_{n}^{l} - β \cdot Penalty ( {w}_{l} )$$

where α and β are parameters that control the relative importance of the uncertainty and penalty terms and where wₗ is the waiting time or latency for the LLM l. Ine reward

$${r}_{n}^{l}$$

represents a tradeoff between performance and cost, defined as:

$${r}_{n}^{l} = \frac{λ}{λ + 1} \cdot {\hat{p}}_{n}^{l} - \frac{1}{λ + 1} \cdot {\hat{c}}_{n}^{l}$$

where λ is a parameter that controls the willingness to prioritize performance over cost, effectively managing the budget.

An uncertainty measurement ensures robustness. The uncertainty may be computed as:

$${Uncertainty}_{l} = {e}_{n}^{T} \cdot {A}_{l}^{- 1} \cdot {e}_{n}$$

where

$${A}_{l}^{- 1}$$

represents the inverse covariance matrix for the l^{th} arm, capturing the uncertainty associated with the prediction.

Considering hardware limitations, it is important to avoid routing queries to LLMs with excessively long waiting times. The penalty for waiting time is therefore given by:

$$Penalty ( {w}_{l} ) = {e}^{\{ γ \cdot ( {w}_{l} - τ ) \}}$$

where γ is a scaling factor and τ represents the maximum tolerable waiting time. The waiting time w for an LLM in arm/includes two components: The initial latency and the token output time. The initial latency is the time required for the LLM to start processing the query, while the token output time is the time taken to generate each token in the response. If the waiting time exceeds t, the penalty increases exponentially, discouraging the selection of such LLMs.

Finally, the LLM with the highest score among the L candidates is selected as the most suitable for the given query:

$$L L {M}_{n}^{c h o s e n} = arg max ( {Score}_{n}^{l} )$$

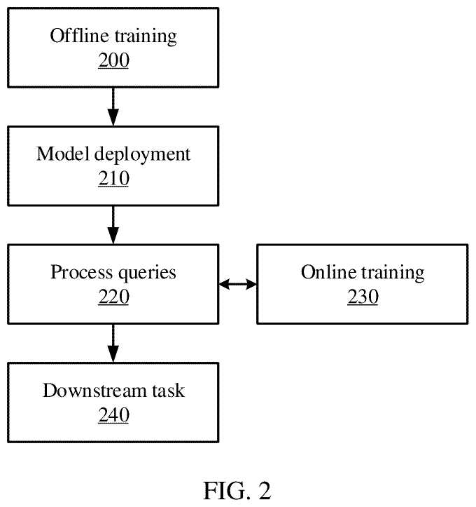

Referring now to FIG. 2, a method of training and using an LLM selector is shown. Block 200 performs offline training of the LLMs 120 and the selector 110. Offline training gathers responses from each LLM 120 in response to training inputs. In this full feedback approach, performance data is collected from all of the arms in the MAB formulation. After offline training 200, the LLMs 120 and the selector 110 may be deployed 210 to a target system, for example by transferring the parameters of the trained models. In embodiments where inference will be performed at the same location as the training, a separate deployment step may be omitted.

Block 220 then uses the trained LLMs 120 and the trained selector 110 to process new queries 100, selecting one of the LLMs 120 to execute each of the new input queries 100. The outputs of the selected LLMs 120 are used to perform a downstream task 240. These outputs are further used to perform online training 230, so that the model can continue to learn and adapt. In online training 230 feedback is only received from the particular LLM 120 which is selected to execute a particular input query 100, referred to herein as partial feedback. Online training adjusts the parameters of the selector 110 to improve its predictions and routing decisions based on real-world usage.

Offline learning 200 includes the parameters of the LLMs 120 and uncertainty estimates. The waiting time is adjusted based on the arm assignment. The parameters

$${θ}_{l}^{P}$$

for the response quality predictors are updated using gradient descent:

$${θ}_{l}^{P} := {θ}_{l}^{P} - {η}_{1} \cdot {∇}_{{θ}_{l}^{P}} L ( {p}_{{}_{n}}^{{}_{{}_{{}_{l}}}} , {\hat{p}}_{n}^{{}_{l}} )$$

where L(.,.) is a loss function such as the mean squared error loss and ηₗ is a hyperparameter that controls learning rate. Similarly, the response length predictor parameters

$${θ}_{l}^{RL}$$

are updated as:

$${θ}_{l}^{R L} := {θ}_{l}^{R L} - {η}_{2} \cdot {∇}_{{θ}_{l}^{R L}} L ( R e s L e {n}_{n}^{l} , ResLe {n}_{n}^{l} ) ,$$

The uncertainty matrices Aₗ are updated incrementally:

$${A}_{l} := {A}_{l} + {e}_{n}^{T} \cdot {e}_{n} .$$

This update accumulates information over time, decreasing the inverse

$${A}_{l}^{- 1} ,$$

indicating increased confidence in predictions.

Online training 230 incrementally updates predictive models and uncertainty matrices using partial feedback from the selected LLMs, adapting to real-time conditions. However, human feedback, often binary (“good” or “not good”), is challenging to refine for training. To address this, an Adaptive Feedback Score

$$( A F {S}_{n}^{l} )$$

is used to capture binary feedback.

The final score for each LLM is updated as:

$${Score}_{n}^{l} = {r}_{n}^{l} + α \cdot {Uncertainty}_{n}^{l} - β \cdot Penalty ( {w}_{l} ) + {κ}_{n}^{l} \cdot {AFS}_{n}^{l}$$

where

$${κ}_{n}^{l}$$

is a confidence factor at time step n. AFS is predicted using a shared neural network:

$$[ A F {S}_{n}^{1} , AF {S}_{n}^{2} , … , {AFS}_{n}^{L} ] = {f}^{A F S} ( {e}_{n} ; {θ}^{A F S} ) ,$$

and the arm with the highest score is selected:

$${l}_{n} = arg {max}_{l} ( {Score}_{n}^{l} ) .$$

Since the network outputs cannot be directly supervised with binary feedback, the Policy Gradient method is used to update θ^{AFS}. The probability of selecting arm l is:

$$π ( l \vert {e}_{n} ; {θ}^{A F S} ) = \frac{exp ( A F {S}_{n}^{l} )}{{\sum }_{k = 1}^{L} exp ( A F {S}_{n}^{k} )}$$

The goal is to maximize the expected reward:

$$J ( {θ}^{A F S} ) = {𝔼}_{l ∼ π ( \cdot \vert {e}_{n} ; {θ}^{A F S} )} [ {r}_{n} ]$$

with gradient:

$${∇}_{{θ}^{A F S}} log π ( {l}_{n} \vert {e}_{n} ; {θ}^{A F S} ) = {∇}_{{θ}^{AFS}} ( AF {S}_{n}^{{l}_{n}} - log {\sum }_{k = 1}^{L} exp ( A F {S}_{n}^{k} ) )$$

The parameters are updated as:

$${θ}^{AFS} := {θ}^{AFS} + {η}_{3} \cdot {∇}_{{θ}^{AFS}} log π ( {l}_{n} \vert {e}_{n} ; {θ}^{AFS} ) \cdot {r}_{n} .$$

The confidence factor

$${κ}_{n}^{l}$$

is adjusted based on the variance of the AFS across the current batch:

$${κ}_{n}^{l} = \frac{1}{{Var}_{n} [ {AFS}_{n}^{l} ] + ϵ}$$

The downstream task 240 may be any task that benefits from the use of an LLM. For example, the downstream task 240 may include processing medical records to identify potential diagnoses or treatments for a patient's health condition. In some cases the downstream task 240 may include a question answering task that accepts queries from a user and generates domain-specific responses. The particular features of the query and the task may influence the selection of the LLM 120 that is used to provide an output, as each LLM may have different capabilities and may be trained on different domain-specific information.

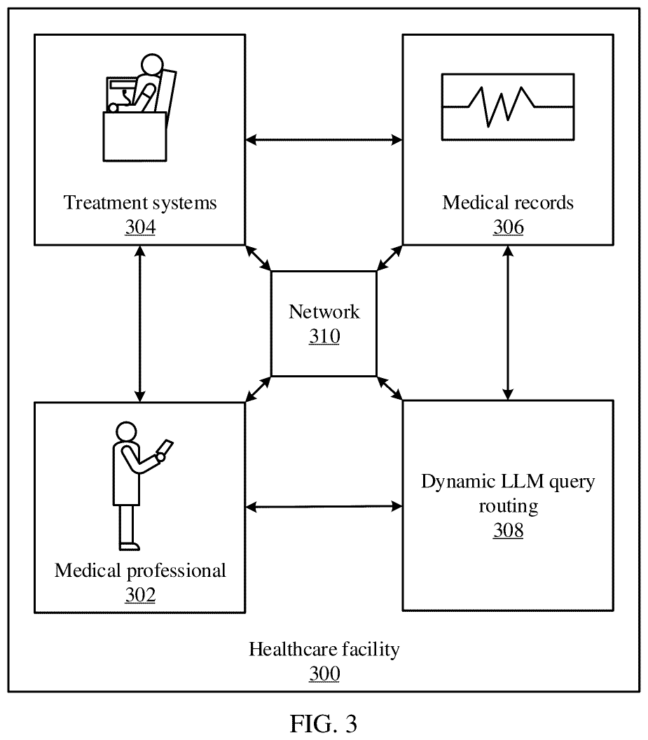

Referring now to FIG. 3, a diagram of dynamic LLM query routing is shown in the context of a healthcare facility 300. Dynamic LLM query routing 308 may be used to select an optimal LLM for processing a query relating to a user's medical condition, for example answering questions about a diagnosis or a patient's medical records 306.

The healthcare facility may include one or more medical professionals 302 who review information extracted from a patient's medical records 306 to determine their healthcare and treatment needs. These medical records 306 may include self-reported information from the patient, test results, and notes by healthcare personnel made to the patient's file. Treatment systems 304 may furthermore monitor patient status to generate medical records 306 and may be designed to automatically administer and adjust treatments as needed.

Dynamic LLM query routing 308 may be used to select an optimal LLM based on the features of an input query and the downstream task. Medical professionals 302 may then make medical decisions about patient healthcare suited to the patient's needs, using the selected LLM to assist. For example, the medical professionals 302 may gather information from the patient's medical records 306 and diagnosis information from the selected LLM to determine a treatment for the patient.

The different elements of the healthcare facility 300 may communicate with one another via a network 310, for example using any appropriate wired or wireless communications protocol and medium. Thus dynamic LLM query routing 308 may receive data from medical professionals 302 and from medical records 306, and may generate outputs that are more accurate and cost-efficient than would be possible otherwise. Dynamic LLM query routing 308 may further coordinate with treatment systems 304 in some cases to automatically administer or alter a treatment in accordance with the downstream task. For example, if the output of the selected LLM determines that a particular treatment may be advisable or harmful, the dynamic LLM query routing 308 may use the output of the selected LLM to automatically send instructions to treatment systems 304 to administer, or halt, the treatment.

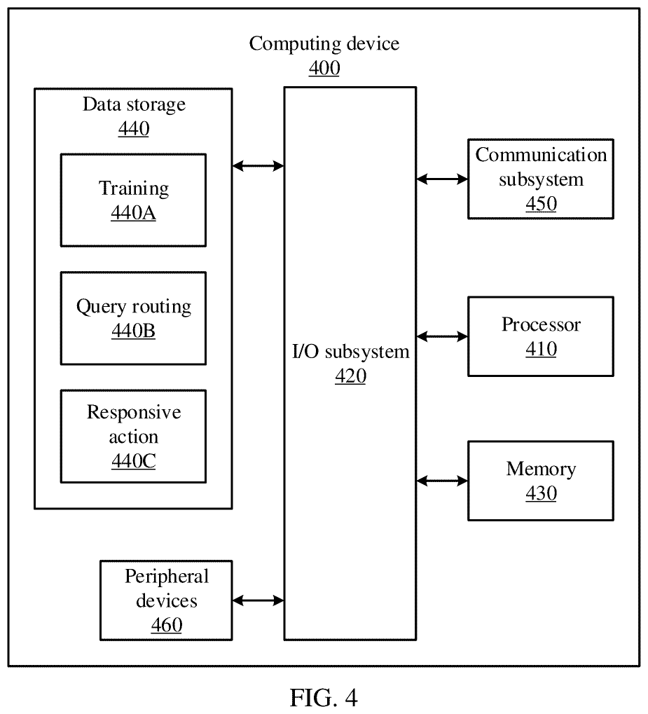

Referring now to FIG. 4, an exemplary computing device 400 is shown, in accordance with an embodiment of the present invention. The computing device 400 is configured to perform dynamic query routing.

The computing device 400 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 400 may be embodied as one or more compute sleds, memory sleds, or other racks, sleds, computing chassis, or other components of a physically disaggregated computing device.

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for training the LLMs, including offline training and online training, 440B for dynamic query routing, and/or 440C for performing responsive actions. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

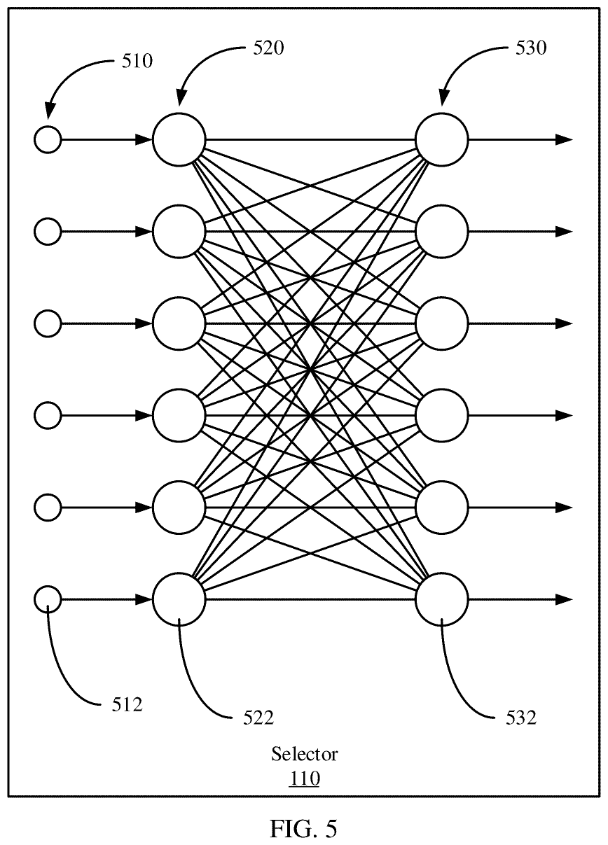

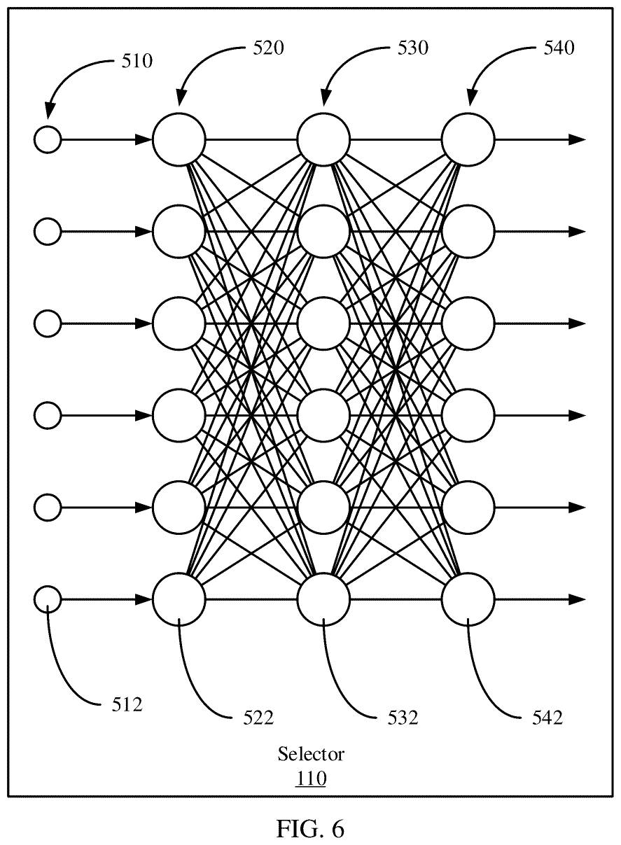

Referring now to FIGS. 5 and 6, exemplary neural network architectures are shown, which may be used to implement parts of the present machine learning models, such as the selector 110. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . , wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 532 in the one or more computation (hidden) layer(s) 530 perform a nonlinear transformation on the input data 512 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
embedding an input query, including contextual information;
predicting performance and cost of executing the input query on each of a plurality of language models, the prediction being performed using a multi-armed bandit approach with each of the language models being represented by a respective arm; and
executing the input query on a selected model of the plurality of models that has a best balance of performance and cost.

2. The method of claim 1, wherein predicting performance includes combining the embedded input query with respective embeddings of states of the plurality of language models.

3. The method of claim 1, wherein predicting cost includes predicting input prompt cost and output response cost.

4. The method of claim 1, further comprising scoring the plurality of language models as:
$${Score}_{n}^{l} = {r}_{n}^{l} + α \cdot {Uncertainty}_{n}^{l} - β \cdot Penalty ( {w}_{l} )$$
wherein l indicates the language model, n indicates the input query,
$${r}_{n}^{l}$$
indicates a combination of the predicted performance and the predicted cost, Uncertaint
$${y}_{n}^{l}$$
is based on a covariance matrix of the language model, Penalty(wₗ) is a time penalty based on latency wₗ, and α and β are balancing parameters.

5. The method of claim 4, wherein the predicted performance
$${\hat{p}}_{n}^{l}$$
and the predicted cost
$${\hat{c}}_{n}^{l}$$
are combined as:
$${r}_{n}^{l} = \frac{λ}{λ + 1} \cdot {\hat{p}}_{n}^{l} - \frac{1}{λ + 1} \cdot {\hat{c}}_{n}^{l}$$
where λ is a parameter that controls willingness to prioritize performance over cost.

6. The method of claim 4, further comprising performing online training that updates an uncertainty of the selected model according to the embedded input query.

7. The method of claim 1, further comprising training a machine learning model encoder that is used in embedding the input query with a composite loss function that has a term to maximize intra-domain similarity and a term to minimize inter-domain similarity.

8. The method of claim 1, further comprising performing a downstream task using an output of the selected model to aid in medical decision making.

9. The method of claim 8, wherein the input query relates to a health condition of a patient and the downstream task includes automatically performing a treatment action on the patient.

10. The method of claim 1, further comprising performing offline training of performance prediction models and cost prediction models, used in predicting the performance and cost respectively, using full feedback from all of the plurality of models.

11. A system, comprising:
a hardware processor; and
a memory that stores a computer program which, when executed by the hardware processor, causes the hardware processor to:
embed an input query, including contextual information;
predict performance and cost of executing the input query on each of a plurality of language models, the prediction being performed using a multi-armed bandit approach with each of the language models being represented by a respective arm; and
execute the input query on a selected model of the plurality of models that has a best balance of performance and cost.

12. The system of claim 11, wherein prediction of performance includes a combination of the embedded input query with respective embeddings of states of the plurality of language models.

13. The system of claim 11, wherein prediction of cost includes prediction of input prompt cost and output response cost.

14. The system of claim 11, wherein the computer program further causes the hardware processor to score the plurality of language models as:
$${Score}_{n}^{l} = {r}_{n}^{l} + α \cdot {Uncertainty}_{n}^{l} - β \cdot Penalty ( {w}_{l} )$$
wherein/indicates the language model, n indicates the input query,
$${r}_{n}^{l}$$
indicates a combination of the predicted performance and the predicted cost, Uncertaint
$${y}_{n}^{l}$$
is based on a covariance matrix of the language model, Penalty(wₗ) is a time penalty based on latency wₗ, and α and β are balancing parameters.

15. The system of claim 14, wherein the predicted performance
$${\hat{p}}_{n}^{l}$$
and the predicted cost
$${\hat{c}}_{n}^{l}$$
are combined as:
$${r}_{n}^{l} = \frac{λ}{λ + 1} \cdot {\hat{p}}_{n}^{l} - \frac{1}{λ + 1} \cdot {\hat{c}}_{n}^{l}$$
where λ is a parameter that controls willingness to prioritize performance over cost.

16. The system of claim 14, wherein the computer program further causes the hardware processor to perform online training that updates an uncertainty of the selected model according to the embedded input query.

17. The system of claim 11, wherein the computer program further causes the hardware processor to train a machine learning model encoder that is used in embedding the input query with a composite loss function that has a term to maximize intra-domain similarity and a term to minimize inter-domain similarity.

18. The system of claim 11, wherein the computer program further causes the hardware processor to perform a downstream task using an output of the selected model to aid in medical decision making.

19. The system of claim 18, wherein the input query relates to a health condition of a patient and the downstream task includes automatically performing a treatment action on the patient.

20. The system of claim 11, wherein the computer program further causes the hardware processor to perform offline training of performance prediction models and cost prediction models, used in predicting the performance and cost respectively, using full feedback from all of the plurality of models.
